(19) United States
(12) Patent Application Publication
(10) Pub. No.: US 2019/0255268 A1
(43) Pub. Date: Aug. 22, 2019

(54) USE OF MULTIPLE SPONTANEOUS BREATH TYPES TO PROMOTE PATIENT VENTILATOR SYNCHRONY

(71) Applicant: Covidien LP, Mansfield, MA (US)

(72) Inventors: Gardner Kimm, Carlsbad, CA (US); Peter Doyle, Vista, CA (US)

(73) Assignee: Covidien LP, Mansfield, MA (US)

(21) Appl. No.: 16/402,394

(22) Filed: May 3, 2019

Related U.S. Application Data
(63) Continuation of application No. 14/477,601, filed on Sep. 4, 2014.
Application No. 14/477,601 is a division of application No. 13/036,825, filed on Feb. 28, 2011, now abandoned.

(51) Int. Cl. A61M 16/00; G16H 40/63

(52) U.S. Cl. CPC A61M 16/0003; G16H 40/63; A61M 16/024; A61M 2205/3303; A61M 2016/0027; A61M 16/0833; A61M 2230/20; A61M 2230/005; A61M 2205/505; A61M 2016/0036; A61M 2230/43

(57) ABSTRACT

The present disclosure combines the advantages of a hybrid mode of ventilation with an automatic determination of an appropriate spontaneous breath type in response to one or more patient based criteria. Specifically, when the ventilator is delivering a spontaneous breath type, a determination may be made as to whether predetermined ventilatory criteria have been met. Based on the determination the ventilator may deliver one of any number of spontaneous breath types.

### INTRODUCTION

Patients who are on mechanical ventilation often experience dyssynchrony with the delivered breaths from the mechanical ventilator. Delays in triggering by the ventilator in response to a patients initial inspiratory effort, mismatching of cycling of ventilator breaths relative to actual patient effort, and mismatches in flow delivery during the inspiratory phase all contribute to patient-ventilator dyssynchrony. It is believed that patients who are on mechanical ventilator support should be allowed to and encouraged to spontaneously breathe. This is not always possible given underlying clinical conditions and the routine use of sedatives in ventilatory assisted patients.

The present disclosure relates to a new method of determining an appropriate spontaneous breath type for use during a hybrid mode of ventilation. A hybrid mode of ventilation encourages and allows the patient to spontaneously breathe while still providing back up support in an effort to maintain at least a minimal level of minute volume. The present disclosure should minimize patient-ventilator dyssynchrony by delivering an appropriate spontaneous breath type in response to one or more ventilatory criteria. By never delivering a patient-initiated mandatory breath, the risk of patient-ventilator dyssynchrony due to flow mismatch and/or inspiratory time mismatch is vastly reduced. Furthermore, by determining an appropriate spontaneous breath type, the present application allows for the movement of a patient from full ventilatory support to full spontaneous ventilation and back based on patient needs. By determining an appropriate spontaneous breath type while providing back up support, the present application reduces ventilator alarms by eliminating the apnea alarm and apnea ventilation function.

As will be discussed in the context of this application, a hybrid mode combines the advantages of mandatory breath types and spontaneous breath types. When using a hybrid mode, the mandatory breath types provide full ventilatory support in the event that the patient is not initiating any breathing effort. Upon detecting a patient effort, the ventilator delivers a spontaneous breath type. If the patient being delivered a spontaneous breath type does not initiate a subsequent effort to breathe within a predetermined backup rate, a mandatory breath type will be delivered. If patient effort is then detected, the ventilator will automatically deliver a spontaneous breath type. In the absence of any patient spontaneous effort, the back up rate with the selected mandatory breath type will be delivered.

The present disclosure combines the advantages of a hybrid mode of ventilation with an automatic determination of an appropriate spontaneous breath type in response to one or more patient based criteria. Specifically, when the ventilator is delivering a spontaneous breath type, a determination may be made as to whether predetermined ventilatory criteria have been met. Based on the determination the ventilator may deliver one of any number of spontaneous breath types.

Embodiments of the present application are directed at a novel method and system for operating a ventilator using multiple spontaneous breath types. In one embodiment, a user selection of two or more spontaneous breath types from a plurality of spontaneous breath types is received. One or more patient respiratory parameters are then monitored during ventilation of a patient. The one or more monitored patient respiratory parameters are then compared to a set of predetermined threshold criteria. Based on the results of the comparison, a breath from the selected spontaneous breath types is delivered.

In another embodiment, a graphical user interface is described for a ventilator to operate in a Hybrid Mode. The graphical user interfaces at least one window associated with the graphical user interface including one or more elements within the at least one window. One of the elements comprises a mode button allowing the selection of one of a plurality of modes. Another element comprises a spontaneous breath type selection element through which a plurality of spontaneous breath types may be selected to be delivered when the ventilator is delivering a breath in response to detection of the trigger criteria.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The following drawing figures, which form a part of this application, are illustrative of described technology and are not meant to limit the scope of the invention as claimed in any manner, which scope shall be based on the claims appended hereto.

FIG. 1 is a diagram illustrating an embodiment of an exemplary ventilator connected to a human patient.

FIG. 2 is a block-diagram illustrating an embodiment of a ventilatory system having a user interface for operating a ventilator using multiple spontaneous breaths in Hybrid Mode.

FIG. 3 is an illustrative flowchart for operating a ventilator using multiple spontaneous breaths.

FIG. 4 is an illustration of a user interface for setting up a new patient attached for ventilation using multiple spontaneous breaths Hybrid Mode.

### DETAILED DESCRIPTION

For the purposes of this disclosure, a “breath” refers to single cycle of inspiration and exhalation delivered with the assistance of a ventilator. The term “breath type” refers to some specific definition or set of rules dictating how the pressure and flow of respiratory gas is controlled by the ventilator during a breath. Breath types may be mandatory breath types (that is, the initiation and termination of the breath is made by the ventilator) or spontaneous (which refers to breath types in which the breath is initiated and terminated by the patient).

A ventilation “mode”, on the other hand, is a set of rules controlling how multiple subsequent breaths should be delivered. Modes may be mandatory, that is controlled by the ventilator, or spontaneous, that is that allow a breath to be delivered or controlled upon detection of a patient's effort to inhale, exhale or both. For example, a simple mandatory mode of ventilation is to deliver one breath of a specified mandatory breath type at a clinician-selected respiratory rate (e.g., one breath every 6 seconds). Until the mode is changed, ventilators will continue to provide breaths of the specified breath type as dictated by the rules defining the mode. This specification describes a third mode, a hybrid mode that provides either a mandatory breath type or a spontaneous breath type depending whether patient inspiratory effort is detected within a predetermined backup rate.

Different spontaneous breath types suit different patient scenarios. Oftentimes, just using one spontaneous breath type may cause the patient to receive an insufficient size of breath (tidal volume). The clinician must become aware that the patient is being delivered an insufficient amount of breath and then change the ventilator settings to an appropriate spontaneous breath type. The present disclosure introduces a method for automatically determining which spontaneous breath type, of a plurality of spontaneous breath types, should be delivered to a patient during Hybrid Mode. Specifically, the ventilator detects that patient measurements have exceeded or fallen below predetermined threshold associated with patient based criteria. When the ventilator determines that the threshold has been crossed, it delivers the appropriate spontaneous breath type, avoiding many of the pitfalls experienced by previous ventilator Hybrid Modes that deliver only a single spontaneous breath type until clinician action is taken.

Ventilator Breath Types A clinician can control patient inspiration and expiration by directing a ventilator to deliver breaths of a specific breath type, usually through the selection of a mode that causes the ventilator to deliver breaths of the desired breath type. Mandatory breath types may be delivered by the ventilator or in response to a patient effort in either mandatory or mandatory/spontaneous modes of ventilation. Spontaneous breath types, on the other hand, require a spontaneously breathing patient in that the initiation is solely based on detection of a patient effort. A ventilator delivering a a spontaneous breath type may trigger and/or cycle in response to a detection of patient effort. Triggering refers to the transition from expiration to inspiration in order to distinguish it from the transition from inspiration to expiration (referred to as cycling).

As discussed above, different patient breath types are characterized by different ventilation waveforms. In general, breath types are characterized primarily by their inhalation phase waveform and by the conditions upon which they trigger and cycle because the exhalation phase in most breaths types is a return to and holding of positive end expiratory pressure (PEEP) from the pressure at the time of cycling. The measured variables of volume, flow, pressure, and time must be calculated to produce the various waveforms. For the purposes of the foregoing disclosure, Volume Support (VS), and Proportional Assist (PA) spontaneous breath types will be discussed, although the reader will note that any breath type now known or later developed may be used.

Volume Support

Volume Support supplies a clinician-selected volume by targeting and controlling the pressure during inhalation. In the VS breath type, a clinician inputs a desired tidal volume, optionally parameters that control the change in pressure and flow between phases, and an exhalation condition such as an exhalation flow threshold. When an inhalation is triggered the ventilator calculates a target pressure from the desired tidal volume and controls to the target pressure. This target pressure is delivered until the exhalation condition is observed, at which point the ventilator cycles to PEEP. If the exhalation condition is not detected within some predetermined period of time (which may be set by the clinician), the ventilator will cycle automatically. In subsequent VS breaths, the difference between the resulting volume and the clinician-set volume is also used to calculate a revised target pressure.

Proportional Assist

The proportional assist (PA) breath type uses automatic estimates of respiratory mechanics (lung/chest wall compliance and airway resistance) to determine the pressure to deliver to a patient. PA differs from previously discussed breath types because ventilator provides pressure, flow, and volume proportional to patient effort. As such, PA breath type can only be used with a patient that is spontaneously triggering breaths and generating some level of inspiratory effort. The amount of pressure provided by the ventilator depends on three factors. First, the amount of pressure corresponds to the flow and volume demanded by the patient effort. Second, the amount of pressure corresponds to a degree of amplification selected by a clinician which determines the extent of ventilator response to patient effort. Third, the amount of pressure corresponds to the estimates of lung/chest wall compliance and airway resistance.

During PA, the ventilator measures the airway flow and pressure and compares these variables to the degree of amplification. When the patient triggers a breath, the ventilator delivers gas in “proportion” to these parameters based on the comparison. As a result, the greater the patient effort detected by the ventilator, the greater the amount of pressure and flow from the ventilator. An advantage of PA over previously discussed spontaneous breath types is the ability to track changes and apply support in response to patient effort.

Multiple Spontaneous Breaths

While VS and PA provide assistance to patients with different ventilatory needs, each spontaneous breath type may no longer be the appropriate spontaneous breath type if the patient's ventilatory needs change. For example, a clinician may notice that a patient who is being administered VS breaths is now struggling to breathe. In this case, the patient may not be being administered enough support and is “pulling” on the ventilator for more. However, VS does not adjust the amount of support delivered based on a patient's pull. In fact, the ventilator will reduce support as the patient's effort increases. On the other hand, a patient being delivered PA breath may suddenly weaken in inspiratory effort. Since PA only delivers support proportionate to the patient demands, the patient exhibiting weak breath effort may not get enough tidal volume while being ventilated using the PA spontaneous breath type.

As discussed above, the present disclosure introduces a method for delivering an appropriate spontaneous breath type based on predetermined patient based criteria. Thus, when the ventilator, operating in a Hybrid Mode, detects that patient measurements have exceeded (that is gone above or fallen below) a predetermined threshold associated with the patient based criteria, it delivers the appropriate spontaneous breath type, avoiding many of the pitfalls experienced by previous ventilator modes that deliver only a single spontaneous breath type until clinician action is taken.

Although the techniques introduced above and discussed in detail below may be implemented for a variety of medical devices, the present disclosure will discuss the implementation of these techniques for use in a mechanical ventilator system. The reader will understand that the technology described in the context of a ventilator system could be adapted for use with other therapeutic equipment having user interfaces, including graphical user interfaces (GUIs), for prompt startup of a therapeutic treatment.

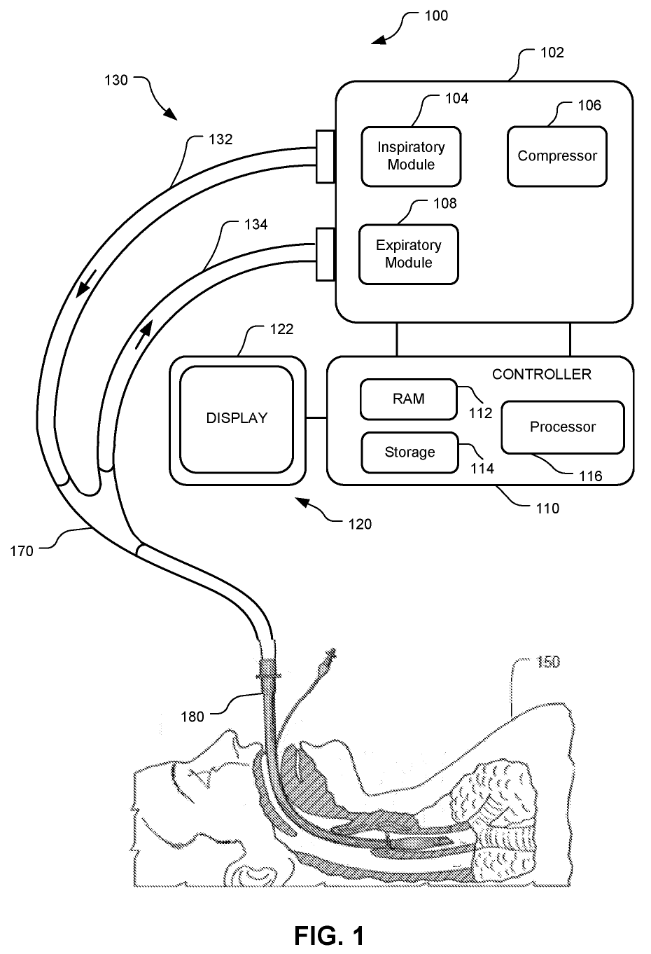

FIG. 1 is a diagram illustrating an embodiment of an exemplary ventilator 100 connected to a human patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 132 and an expiratory limb 134 of the ventilation tubing system 130.

Pneumatic system 102 may be configured in a variety of ways. In the present example, system 102 includes an expiratory module 108 coupled with the expiratory limb 134 and an inspiratory module 104 coupled with the inspiratory limb 132. Compressor 106 or other source(s) of pressurized gases (e.g., air, oxygen, and/or helium) is coupled with inspiratory module 104 to provide a gas source for ventilatory support via inspiratory limb 132.

The pneumatic system 102 may include a variety of other components, including mixing modules, valves, sensors, tubing, accumulators, filters, etc. Controller 110 is operatively coupled with pneumatic system 102, signal measurement and acquisition systems, and an operator interface 120 that may enable an operator to interact with the ventilator 100 (e.g., change ventilator settings, select operational modes, view monitored parameters, etc.). Controller 110 may include memory 112, one or more processors 116, storage 114, and/or other components of the type commonly found in command and control computing devices. In the depicted example, operator interface 120 includes a display 122 that may be touch-sensitive and/or voice-activated, enabling the display to serve both as an input and output device.

The memory 112 includes non-transitory, computer-readable storage media that stores software that is executed by the processor 116 and which controls the operation of the ventilator 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

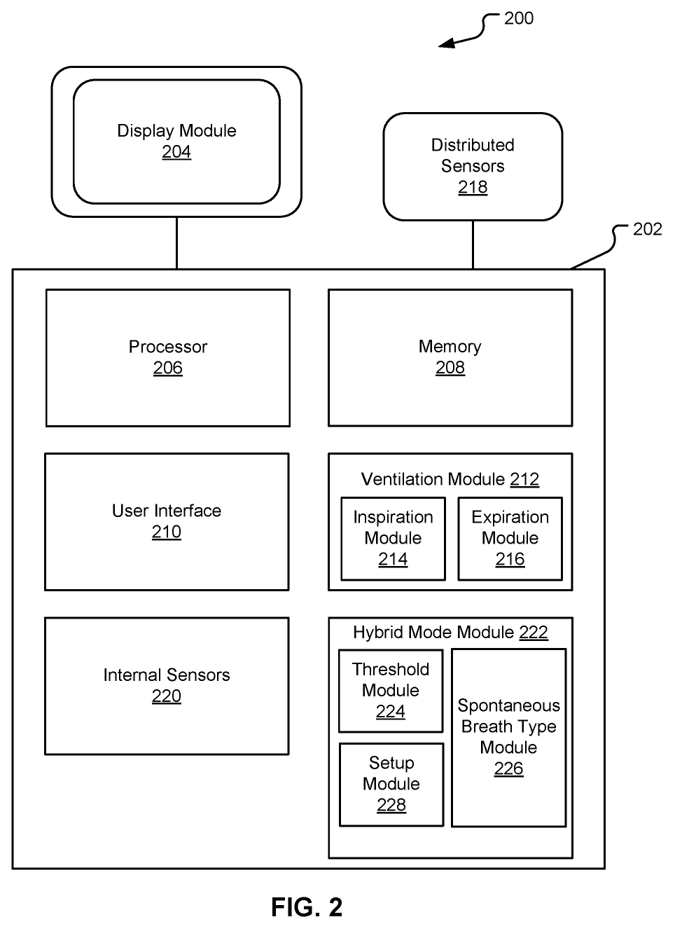

FIG. 2 is a block-diagram illustrating an embodiment of a ventilatory system for implementing Hybrid Mode ventilation.

Ventilatory system 200 includes ventilator 202 with its various modules and components. That is, ventilator 202 may further include, inter alia, memory 208, one or more processors 206, user interface 210, and ventilation module 212 (which may further include an inspiration module 214 and an expiration module 216). Memory 208 is defined as described above for memory 112. Similarly, the one or more processors 206 are defined as described above for one or more processors 116. Processors 206 may further be configured with a clock whereby elapsed time may be monitored by the system 200.

The ventilatory system 200 may also include a display module 204 communicatively coupled to ventilator 202. Display module 204 provides various input screens, for receiving clinician input, and various display screens, for presenting useful information to the clinician. The display module 204 is configured to communicate with user interface 210 and may include a graphical user interface (GUI). The GUI may be an interactive display, e.g., a touch-sensitive screen or otherwise, and may provide various windows and elements for receiving input and interface command operations. Alternatively, other suitable means of communication with the ventilator 202 may be provided, for instance by a wheel, keyboard, mouse, or other suitable interactive device. Thus, user interface 210 may accept commands and input through display module 204. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed to the clinician in the form of graphs, wave representations, pie graphs, or other suitable forms of graphic display. For example, a settings screen may be displayed on the GUI and/or display module 204 to configure hybrid mode ventilation.

Ventilation module 212 may further include an inspiration module 214 configured to deliver gases to the patient according to prescribed ventilatory settings. Specifically, inspiration module 214 may correspond to the inspiratory module 104 or may be otherwise coupled to source(s) of pressurized gases (e.g., air, oxygen, and/or helium), and may deliver gases to the patient. Inspiration module 214 may be configured to provide ventilation according to various ventilatory breath types. As discussed above, these breath types may include VS and PA. Thus, the ventilation module 212 includes the algorithms and computer-readable instructions necessary to provide any desired breath type.

Ventilation module 212 may further include an expiration module 216 configured to release gases from the patient's lungs according to prescribed ventilatory settings. Specifically, expiration module 216 may correspond to expiratory module 108 or may otherwise be associated with and/or controlling an expiratory valve for releasing gases from the patient. By way of general overview, a ventilator may initiate expiration based on lapse of an inspiratory time setting or other cycling criteria set by the clinician or derived from ventilator settings (e.g., detecting delivery of prescribed tidal volume or prescribed pressure). Upon initiating the expiratory phase, expiration module 216 may allow the patient to exhale by opening an expiratory valve. As such, expiration is passive, and the direction of airflow is governed by the pressure gradient between the patient's lungs (higher pressure) and the ambient surface pressure (lower pressure). Although expiratory flow is passive, it may be regulated by the ventilator based on the size of the expiratory valve opening.

According to some embodiments, the inspiration module 214 and/or the expiration module 216 may be configured to synchronize ventilation with a spontaneously-breathing, or triggering, patient. Specifically, the ventilator may detect patient effort via a pressure-monitoring method, a flow-monitoring method, direct or indirect measurement of nerve impulses, or any other suitable method. Sensing devices may be either internal or distributed and may include any suitable sensing device, as described further herein. In addition, the sensitivity of the ventilator to changes in pressure and/or flow may be adjusted such that the ventilator may properly detect the patient effort, i.e., the lower the pressure or flow change setting the more sensitive the ventilator may be to patient triggering.

According to embodiments, a pressure-triggering method may involve the ventilator monitoring the circuit pressure, as described above, and detecting a slight drop in circuit pressure. The slight drop in circuit pressure may indicate that the patient's respiratory muscles are creating a slight negative pressure gradient between the patient's lungs and the airway opening in an effort to inspire. The ventilator may interpret the slight drop in circuit pressure as patient effort and may consequently initiate inspiration by delivering respiratory gases.

Alternatively, the ventilator may detect a flow-triggered event. Specifically, the ventilator may monitor the circuit flow, as described above. If the ventilator detects a slight drop in flow during exhalation, this may indicate, again, that the patient is attempting to inspire. In this case, the ventilator is detecting a drop in bias flow (or baseline flow) attributable to a slight redirection of gases into the patient's lungs (in response to a slightly negative pressure gradient as discussed above). Bias flow refers to a constant flow existing in the circuit during exhalation that enables the ventilator to detect expiratory flow changes and patient triggering. For example, while gases are generally flowing out of the patient's lungs during expiration, a drop in flow may occur as some gas is redirected and flows into the lungs in response to the slightly negative pressure gradient between the patient's lungs and the body's surface. Thus, when the ventilator detects a slight drop in flow below the bias flow by a predetermined threshold amount (e.g., 2 L/min below bias flow), it may interpret the drop as a patient trigger and may consequently initiate inspiration by delivering respiratory gases.

The ventilatory system 200 may also include one or more distributed sensors 218 communicatively coupled to ventilator 202. Distributed sensors 218 may communicate with various components of ventilator 202, e.g., ventilation module 212, internal sensors 220, Hybrid Mode module 222, threshold module 224, and any other suitable components and/or modules. Distributed sensors 218 may detect changes in patient measurements indicative of crossing a Hybrid Mode threshold, for example. Distributed sensors 218 may be placed in any suitable location, e.g., within the ventilatory circuitry or other devices communicatively coupled to the ventilator. For example, sensors may be affixed to the ventilatory tubing or may be imbedded in the tubing itself. According to some embodiments, sensors may be provided at or near the lungs (or diaphragm) for detecting a pressure in the lungs. Additionally or alternatively, sensors may be affixed or imbedded in or near wye-fitting 170 and/or patient interface 180, as described above.

Distributed sensors 218 may further include pressure transducers that may detect changes in circuit pressure (e.g., electromechanical transducers including piezoelectric, variable capacitance, or strain gauge). Distributed sensors 218 may further include various flow sensors for detecting airflow (e.g., differential pressure pneumotachometers). For example, some flow sensors may use obstructions to create a pressure decrease corresponding to the flow across the device (e.g., differential pressure pneumotachometers) and other flow sensors may use turbines such that flow may be determined based on the rate of turbine rotation (e.g., turbine flow sensors). Alternatively, sensors may utilize optical or ultrasound techniques for measuring changes in ventilatory parameters. A patient's blood parameters or concentrations of expired gases may also be monitored by sensors to detect physiological changes that may be used as indicators to study physiological effects of ventilation, wherein the results of such studies may be used for diagnostic or therapeutic purposes. Indeed, any distributed sensory device useful for monitoring changes in measurable parameters during ventilatory treatment may be employed in accordance with embodiments described herein.

Ventilator 202 may further include one or more internal sensors 220. Similar to distributed sensors 218, internal sensors 220 may communicate with various components of ventilator 202, e.g., ventilation module 212, internal sensors 220, Hybrid Mode module 222, threshold module 224, and any other suitable components and/or modules. Internal sensors 220 may employ any suitable sensory or derivative technique for monitoring one or more parameters associated with the ventilation of a patient. However, the one or more internal sensors 220 may be placed in any suitable internal location, such as, within the ventilatory circuitry or within components or modules of ventilator 202. For example, sensors may be coupled to the inspiratory and/or expiratory modules for detecting changes in, for example, circuit pressure and/or flow. Specifically, internal sensors may include pressure transducers and flow sensors for measuring changes in circuit pressure and airflow. Additionally or alternatively, internal sensors may utilize optical or ultrasound techniques for measuring changes in ventilatory parameters. For example, a patient's expired gases may be monitored by internal sensors to detect physiologic changes indicative of the patient's condition and/or treatment. Indeed, internal sensors may employ any suitable mechanism for monitoring parameters of interest in accordance with embodiments described herein.

As should be appreciated, ventilatory parameters are highly interrelated and, according to embodiments, may be either directly or indirectly monitored. That is, parameters may be directly monitored by one or more sensors, as described above, or may be indirectly monitored by derivation.

Ventilator 200 may further include Hybrid Mode module 222. Hybrid Mode module is activated when a clinician indicates that the ventilator should run in Hybrid Mode. Hybrid Mode allows a ventilator to be programmed to use a first breath type in response to a spontaneous trigger (that is, when the ventilator detects that the patient is trying to inhale) and a second breath type in response to a mandatory trigger event (e.g., upon the expiration of a timer). The Hybrid Mode module controls when and how breath types are delivered.

The Hybrid Mode module 222 is communicatively coupled to both threshold module 224 and spontaneous breath type module 226. Threshold module 224 is configured to detect when patient measurements have crossed a predetermined threshold indicative of a patient's effort to initiate a breath. The predetermined threshold serves as an indicator that the Hybrid Mode module 222 should deliver the breath type selected for spontaneous breathing. For example, a tidal volume threshold may be set for 80%, an inspiratory pressure threshold may be set for 12 cm H₂O, and a rapid shallow breathing index threshold may be set to 100. As will be appreciated, these are some of many thresholds that may be crossed, all of which are within the scope of the present disclosure. When a threshold is exceeded, the threshold module 224 communicates an exceeded threshold to the spontaneous breath type module 226.

The Hybrid Mode module 222 is also communicatively coupled to the spontaneous breath type module 226. Upon indication that the ventilator should deliver a spontaneous breath type, the spontaneous breath type module 226 communicates to the ventilator an appropriate spontaneous breath type for delivery. The spontaneous breath type module 226 determines which spontaneous breath type is appropriate for the patient through communication with the threshold module 224. For example, the threshold module 224 may communicate to the spontaneous breath type module 226 that a threshold has been crossed. The spontaneous breath type module 226 may then process this information to determine an appropriate spontaneous breath type. For example, if the tidal volume is less than 80% of a set threshold, the spontaneous breath type module 226 may communicate to the ventilator that the spontaneous breath type should be VS instead of PA. If the inspiratory pressure drops below 12 cm H₂O, the spontaneous breath type module 226 may indicate to the ventilator that PA should be used instead of VS. If the rapid shallow breathing index is greater than 100, the spontaneous breath type module 226 may communicate to the ventilator that VS should be used as the spontaneous breath type instead of PA. As will be appreciated, these thresholds are exemplary and many different thresholds are contemplated within the scope of the present disclosure. Determining an appropriate spontaneous breath type will be discussed in further detail below.

The Hybrid Mode module 222 is also communicatively coupled to setup module 228. Setup module 228 is coupled with display module 204 to provide configuration options for Hybrid Mode at setup. Specifically, setup module 228 provides display module 204 with two Hybrid Mode configuration options. The first configuration option is “Easy Mode” and configures the ventilator to operate in Hybrid Mode using preselected mandatory and spontaneous breath types. In one embodiment, “Easy Mode” automatically designates PA and VS as the spontaneous breath types. The second configuration option provided by the setup module 226 is “Config Mode.” The “Config Mode” is intended for the more sophisticated user that wants maximum control over the ventilator. When the setup module 226 receives an indication that the clinician has chosen “Config Mode,” it provides a list of all available spontaneous breath types for selection by the clinician. The clinician may then select one or more spontaneous breath types for delivery to the patient. The setup module 228 communicates the selected breath types to the threshold module 224 and Hybrid Mode module 222.

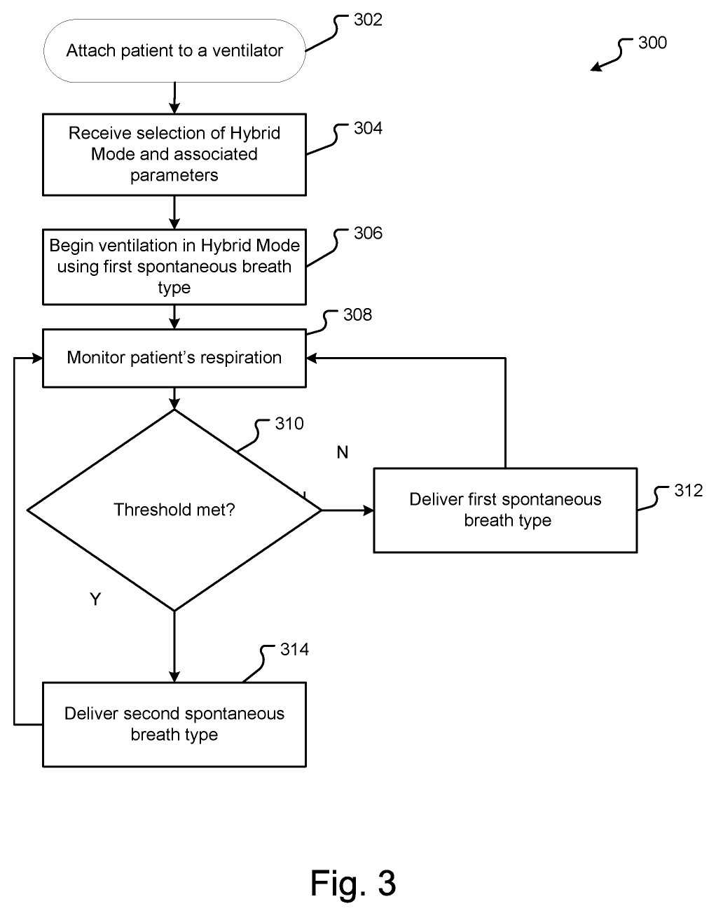

FIG. 3 represents an illustrative flow 300 for operating a ventilator in Hybrid Mode. At attach operation 302, a patient is attached to a ventilator. Once the patient is properly attached to the ventilator, flow proceeds to receive operation 304.

At receive operation 304, an indication is received that the ventilator is set to operate in Hybrid Mode. Such an indication may come from a graphical user interface that displays “Hybrid Mode” as a selectable element. The indication that the ventilator is set to operate in Hybrid Mode is accompanied by the breath type parameters to be used during spontaneous breaths. In one embodiment, the breath type parameters are preselected as the clinician has chosen to setup Hybrid Mode using an “Easy Mode.” For example, setting up Hybrid Mode with “Easy Mode” may communicate that PA and VS spontaneous breath types should be used. Alternatively, the breath type parameters are designated by a clinician using a “Config Mode.” If the clinician sets up Hybrid Mode using “Config Mode,” any available spontaneous breath type(s) may be selected. For the purposes of this discussion, PA and VS will be described as the selected spontaneous breath types. However, it will be appreciated that any spontaneous breath types may be utilized for the purposes of the present application. The spontaneous breath types are communicated alongside the indication that the ventilator is set to operate in Hybrid Mode and flow proceeds to begin operation 306.

At begin operation 306, the ventilator begins ventilation in Hybrid Mode by delivering a first spontaneous breath type, unless no spontaneous efforts are detected, in which case a mandatory breath is delivered. In one embodiment, the first spontaneous breath type is PA. For example, a patient exhibiting weak inspiration effort after waking up from surgery may be ventilated using PA. Flow then proceeds to a monitor operation 308.

At the monitor operation 308, the ventilator monitors patient based criteria. As discussed above, Hybrid Mode operates on a breath to breath basis. As a result, patient measurements are monitored per breath. The monitoring is done by any of the internal and/or distributed sensors discussed above. The sensors can measure any relevant patient based criteria including but not limited to work of breathing, carbon dioxide output, inspiratory pressure, expiratory pressure, inspiratory volume, expiratory volume, body weight, respiratory rate, minute ventilation and target pressure. These patient based criteria are used by the ventilator to determine whether the patient is being administered the appropriate breath type. In one embodiment, the sensors can only detect patient effort during exhalation. In another embodiment, the sensors can detect patient effort during both inspiration and exhalation. Once the patient measurements have been monitored, flow proceeds to detect operation 310.

At detect operation 310, a determination is made as to whether a threshold associated with the patient based criteria has been crossed. For example, a determination may be made as to whether a patient is displaying an effort that is too weak. If the patient effort is not too weak, then a determination may be made that the appropriate spontaneous breath type is being delivered, the first spontaneous breath type is delivered again at operation 312 and flow returns to monitor operation 308. However, the ventilator may be currently delivering the patient PA spontaneous breath type but the patient is displaying weak effort. As a result the patient is not receiving enough volume and the PA spontaneous breath type may no longer be appropriate. If a determination is made that the appropriate spontaneous breath type is not being delivered, flow proceeds to deliver operation 314.

At deliver operation 314, a second spontaneous breath type is delivered to the patient. For example, the patient may be delivered a VS spontaneous breath type. By delivering the patient a VS spontaneous breath type, the patient will be delivered a set volume, helping the patient who is exhibiting weak inspiratory effort. Flow then returns to monitor operation 308.

In embodiments of the method 300 may utilize different spontaneous breath types than PA and VS. Moreover, any number of spontaneous breath types may be administered in combination within the scope of the present disclosure. For example, a clinician may select more than two spontaneous breath types. Furthermore if, at any time, the patient does not exhibit an inspiratory effort within a set respiratory rate (or backup rate) the ventilator may administer a mandatory breath, as will be appreciated within the context of Hybrid Mode.

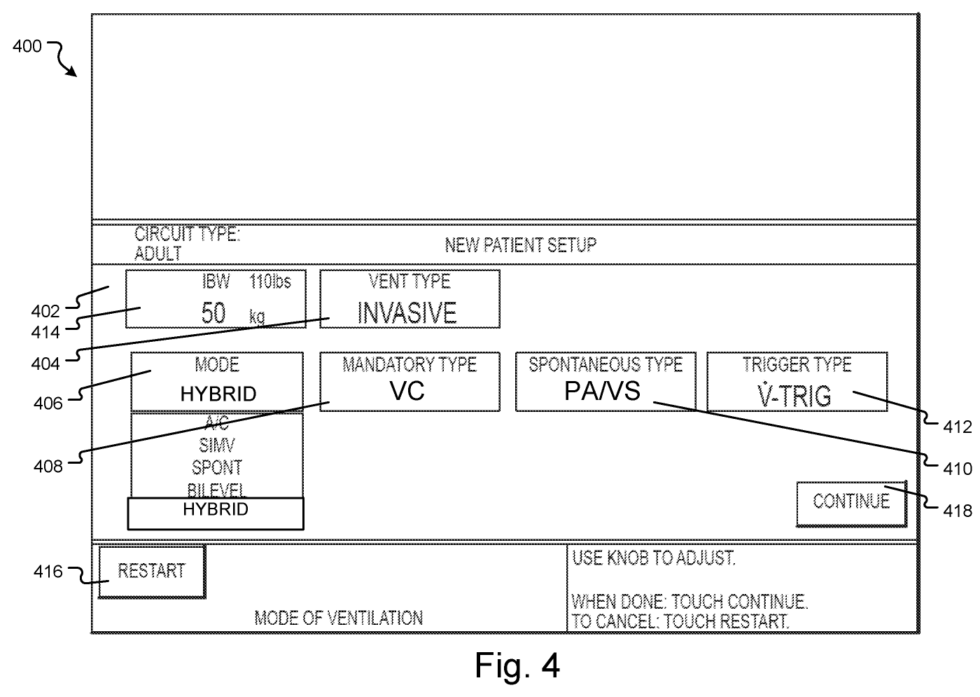

FIG. 4 is an illustration of a user interface for setting up a new patient attached for ventilation using Hybrid Mode.

For the purposes of the foregoing discussion, the user interfaces may be accessed via any suitable means, for example via a main ventilatory user interface on display module. As illustrated, the user interfaces may provide one or more windows for display and one or more elements for selection and/or input. Windows may include one or more elements and, additionally, may provide graphical displays, instructions, or other useful information to the clinician. Elements may be displayed as buttons, tabs, icons, toggles, or any other suitable visual access element, etc., including any suitable element for input selection or control.

According to one embodiment, as illustrated by FIG. 4, new patient setup interface 400 may include new patient setup window 402. New patient setup window 402 may include one or more selectable elements to configure new patient setup. New patient setup window 402 may include a Vent Type button 404. Vent Type button 404 allows a clinician to select a type of ventilation for the patient. In one embodiment, when the clinician selects the Vent Type button 404 a pull down menu appears underneath the Vent Type button 404 displaying vent type options (not depicted). The clinician can then select one of the vent type options to set as the Vent Type. The vent type options may include invasive and non-invasive. These vent type options correspond to the way that the patient was attached to the ventilator as discussed in detail with reference to FIG. 1. As will be appreciated, when a vent type option is selected, it is displayed in the Vent Type button 404 as depicted in FIG. 4.

New patient setup window 402 may be further configured to include a Mode button 406. Like the Vent Type button 404, when a clinician selects the Mode button 406, a pull down menu appears under the Mode button 406. The pull down menu displays various modes options for selection. In one embodiment, the pull down menu includes a Hybrid option. In another embodiment, the pull down menu includes Hybrid-Easy and Hybrid-Config options (not illustrated). As discussed above, the Hybrid-Easy option may be selected for a preconfigured Hybrid Mode ventilator setup. The Hybrid-Config option may be selected by a user who wants to specify each spontaneous breath type utilized during Hybrid Mode. As will be appreciated, when a mode option is selected, it is displayed in the Mode button 406 as depicted in FIG. 4.

The new patient setup window 402 may be further configured to include a Mandatory Type button 408. When the clinician selects the Mandatory Type button 408 a pull down menu appears under the Mandatory Type button 408. The pull down menu displays various mandatory type options for selection. The mandatory type options are mandatory breath types. As will be appreciated, when a mandatory type option is selected, it is displayed in the Mandatory Type button 408 as depicted in FIG. 4.

The new patient setup window 402 may be further configured to include a Spontaneous Type button 410. When the clinician selects the Spontaneous Type button 410 a pull down menu appears under the Spontaneous Type button 410. The pull down menu displays various spontaneous type options for selection. In one embodiment, if the Mode button 406 is set to Hybrid-Easy, the Mandatory Type button 408 is automatically set to PA or VS. In another embodiment, if the ventilator Mode button 406 is set to Hybrid-Config, the clinician can choose from any of the spontaneous breath types. As will be appreciated, when a spontaneous type option is selected, it is displayed in the Spontaneous Type button 410 as depicted in FIG. 4.

The new patient setup window 402 may be further configured to include a Trigger Type button 412. When the clinician selects the Trigger Type button 412 a pull down menu appears under the Trigger Type button 412. The pull down menu displays various trigger type options for selection. These trigger types may include a flow trigger and a pressure trigger. As will be appreciated, the selected trigger type determines the patient measurement(s) used to determine if a patient is spontaneously triggering. In one embodiment, if the ventilator Mode button 406 is set to Hybrid-Easy, the Trigger Type button 408 is automatically set to flow trigger. In another embodiment, if the ventilator Mode button 406 is set to Hybrid-Config, the clinician can choose from any of available trigger types such as pressure, flow, volume, patient effort, etc. As will be appreciated, when a trigger type option is selected, it is displayed in the Trigger Type button 412 as depicted in FIG. 4.

The new patient setup window 402 may include various other selectable elements. For example, the window may include an Ideal Body Weight button 414 and a restart button 416. Like the other buttons discussed above with reference to FIG. 4, the Ideal Body Weight button 414 may be selected to change the Ideal Body Weight setting of a patient. The restart button 416 may also be selected to restart the ventilator.

Once a clinician is satisfied with the settings displayed on the new patient setup window 402, the clinician may select the continue button 416 to configure the ventilator with the displayed settings. When the continue button 416 has been selected, the ventilator may display a ventilator settings interface.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components, in various combinations of hardware and software, and individual functions can be distributed among software applications at either the client or server level. In this regard, any number of the features of the different embodiments described herein may be combined into one single embodiment and alternative embodiments having fewer than or more than all of the features herein described are possible.

While various embodiments have been described for purposes of this disclosure, various changes and modifications may be made which are well within the scope of the present technology. Numerous other changes may be made which will readily suggest themselves to those skilled in the art and which are encompassed in the spirit of the disclosure and as defined in the appended claims.

1.-18. (canceled)

19. A method for operating a ventilator, the method comprising:
receiving a selection of a Hybrid Mode for ventilation of a patient, wherein the Hybrid Mode is configured to automatically deliver a breath of a mandatory breath type when the patient fails to initiate a breathing effort and automatically deliver a breath of a spontaneous breath type when the patient initiates a breathing effort;
configuring the Hybrid Mode with at least two spontaneous breath types of a plurality of spontaneous breath types;
in response to detecting a first breathing effort, automatically delivering a first breath according to a first spontaneous breath type of the at least two spontaneous breath types;
monitoring at least one respiratory parameter, wherein the at least one respiratory parameter is selected from the group consisting of: tidal volume and patient effort;
comparing at least one measurement of the at least one respiratory parameter to a threshold; and
based on the comparing and in response to detecting a second breathing effort, automatically delivering a second breath according to a second spontaneous breath type of the at least two spontaneous breath types.

20. The method of claim 19, wherein the at least one respiratory parameter is tidal volume, and wherein the comparing indicates that the tidal volume of the first breath was less than the threshold.

21. The method of claim 19, wherein the at least one respiratory parameter is patient effort, and wherein the comparing indicates that the second breathing effort was less than the threshold.

22. The method of claim 19, wherein the at least one respiratory parameter is tidal volume, and wherein the threshold is 80% of a set tidal volume.

23. The method of claim 19, wherein the at least one respiratory parameter is determined based on one or more of: a carbon dioxide level, inspiratory pressure, expiratory pressure, inspiratory volume, expiratory volume, body weight, minute ventilation, or lung/chest wall compliance.

24. The method of claim 19, wherein the plurality of spontaneous breath types comprise: proportional assist (PA), Pressure Support (PS), and volume support (VS) breath types.

25. The method of claim 19, wherein threshold is specific to the at least one respiratory parameter.

26. A ventilator comprising:
a processor; and
a memory storing computer-readable instructions that when executed by the processor cause the ventilator to:
receive a selection of a Hybrid Mode for ventilation of a patient, wherein the Hybrid Mode is configured to automatically deliver a breath of a mandatory breath type when the patient fails to initiate a breathing effort and automatically deliver a breath of a spontaneous breath type when the patient initiates a breathing effort;
configure the Hybrid Mode with at least two spontaneous breath types of a plurality of spontaneous breath types;
in response to detecting a first breathing effort, automatically deliver a first breath according to a first spontaneous breath type of the at least two spontaneous breath types;
measure a tidal volume of the first breath;
compare the tidal volume to a threshold; and
based on the comparing and in response to detecting a second breathing effort, automatically deliver a second breath according to a second spontaneous breath type of the at least two spontaneous breath types.

27. The system of claim 26, wherein the Hybrid Mode delivers a mandatory breath if a breathing effort is not detected within a set time based on a desired respiratory rate.

28. The system of claim 26, wherein the comparing indicates that the tidal volume of the first breath was less than the threshold.

29. The system of claim 26, wherein the threshold is 80% of a set tidal volume.

30. The system of claim 26, wherein the tidal volume is determined based on one or more of: a carbon dioxide level, inspiratory pressure, expiratory pressure, inspiratory volume, expiratory volume, body weight, minute ventilation, or lung/chest wall compliance.

31. The system of claim 26, wherein the plurality of spontaneous breath types comprise: proportional assist (PA), Pressure Support (PS), and volume support (VS) breath types.

32. The system of claim 26, wherein the first spontaneous breath type is proportional assist (PA), and wherein the second spontaneous breath type is Pressure Support (PS).

33. The system of claim 26, wherein the first spontaneous breath type is Pressure Support (PS), and wherein the second spontaneous breath type is proportional assist (PA).

34. A computer-readable medium storing computer-readable instructions that when executed by a processor cause a ventilator to:
receive a selection of a Hybrid Mode for ventilation of a patient, wherein the Hybrid Mode is configured to automatically deliver a breath of a mandatory breath type when the patient fails to initiate a breathing effort and automatically deliver a breath of a spontaneous breath type when the patient initiates a breathing effort;
configure the Hybrid Mode with at least two spontaneous breath types of a plurality of spontaneous breath types;
in response to detecting a first breathing effort, automatically deliver a first breath according to a first spontaneous breath type of the at least two spontaneous breath types;
monitor at least one respiratory parameter, wherein the at least one respiratory parameter is selected from the group consisting of: tidal volume and patient effort;
compare at least one measurement of the at least one respiratory parameter to a threshold; and
based on the comparing and in response to detecting a second breathing effort, automatically deliver a second breath according to a second spontaneous breath type of the at least two spontaneous breath types.

35. The computer-readable medium of claim 34, wherein the at least one respiratory parameter is tidal volume, and wherein the comparing indicates that the tidal volume of the first breath was less than the threshold.

36. The computer-readable medium of claim 34, wherein the at least one respiratory parameter is patient effort, and wherein the comparing indicates that the second breathing effort was less than the threshold.

37. The computer-readable medium of claim 34, wherein the Hybrid Mode delivers a mandatory breath if a breathing effort is not detected within a set time based on a desired respiratory rate.

38. The computer-readable medium of claim 34, wherein the first spontaneous breath type is proportional assist (PA), and wherein the second spontaneous breath type is Pressure Support (PS).
